SEQUENCE LISTING (25 sequences)

SEQ ID NO: 1 - protein, 38 aa, Mus musculus
GNMTDSFTKAEASILPSITNQSGPNSTLIISNSSLEEE

SEQ ID NO: 2 - protein, 16 aa, Mus musculus
KVLTSFTNKELQAYAK

SEQ ID NO: 3 - protein, 14 aa, Mus musculus
SRDDDHETKRQNCN

SEQ ID NO: 4 - protein, 43 aa, Mus musculus
KGGNMTDSFTKAEASILPSITNQSGPNSTLIISNSSLEEEGKK

SEQ ID NO: 5 - protein, 21 aa, Mus musculus
KGKVLTSFTNKELQAYAKGKK

SEQ ID NO: 6 - protein, 19 aa, Mus musculus
KGSRDDDHETKRQNCNGKK

SEQ ID NO: 7 - protein, 47 aa, Homo sapiens
GEMTDIFANAGNLEDLMSNITNRSDINDTGFFMNLEEDMTRYAYYYS

SEQ ID NO: 8 - protein, 18 aa, Homo sapiens
GEMTDIFANAGNLEDLMS

SEQ ID NO: 9 - protein, 14 aa, Homo sapiens
NITNRSDINDTGFF

SEQ ID NO: 10 - protein, 15 aa, Homo sapiens
MNLEEDMTRYAYYYS

SEQ ID NO: 11 - protein, 25 aa, Homo sapiens
FSKIIGVFTRIDDPETKRQNSNLFS

SEQ ID NO: 12 - protein, 18 aa, Homo sapiens
FTRIDDPETKRQNSNLFS

SEQ ID NO: 13 - protein, 17 aa, Homo sapiens
FRFGAYLVAHKLMSFED

SEQ ID NO: 14 - protein, 52 aa, Homo sapiens
KGGEMTDIFANAGNLEDLMSNITNRSDINDTGFFMNLEEDMTRYAYYYSGKK

SEQ ID NO: 15 - protein, 23 aa, Homo sapiens
KGGEMTDIFANAGNLEDLMSGKK

SEQ ID NO: 16 - protein, 19 aa, Homo sapiens
KGNITNRSDINDTGFFGKK

SEQ ID NO: 17 - protein, 20 aa, Homo sapiens
KGMNLEEDMTRYAYYYSGKK

SEQ ID NO: 18 - protein, 30 aa, Homo sapiens
KGFSKIIGVFTRIDDPETKRQNSNLFSGKK

SEQ ID NO: 19 - protein, 23 aa, Homo sapiens
KGFTRIDDPETKRQNSNLFSGKK

SEQ ID NO: 20 - protein, 22 aa, Homo sapiens
KGFRFGAYLVAHKLMSFEDGKK

SEQ ID NO: 21 - protein, 16 aa, Mus musculus
GGNMTDSFTKAEASGK

SEQ ID NO: 22 - protein, 15 aa, Mus musculus
GLPSITNQSGPNSGK

SEQ ID NO: 23 - protein, 15 aa, Mus musculus
GTLIISNSSLEEEGK

SEQ ID NO: 24 - protein, 19 aa, Mus musculus
GKVLTSFTNKELQAYAKGK

SEQ ID NO: 25 - protein, 17 aa, Mus musculus
GSRDDDHETKRQNCNGK

(19) United States
(12) Patent Application Publication
(10) Pub. No.: US 2005/0255561 A1
(43) Pub. Date: Nov. 17, 2005

(54) Therapeutic vaccine targeted against P-glycoprotein 170 for inhibiting multidrug resistance in the treatment of cancers

(76) Inventors: Pierre-Francois Tosi, Reims (FR); Claudie Madoulet, Reims (FR); Claude Yves Nicolau, Newton, MA (US); David T. Hickman, Strasbourg Cedex (FR)

Correspondence Address: JOYCE VON NATZMER, 4615 NORTH PARK AVENUE, SUITE 919, CHEVY CHASE, MD, 20815, US

(21) Appl. No.: 11/059,633

(22) Filed: Feb. 16, 2005

Related U.S. Application Data
(63) Continuation-in-part of application No. 10/902,276, filed on Jul. 30, 2004.
Application No. 10/902,276 is a continuation of application No. PCT/EP04/08330, filed on Jul. 25, 2004.

(30) Foreign Application Priority Data
Jul. 25, 2003 (FR) FR0309188

(51) Int. Cl. C07H 21/04; C12P 21/06; C07K 14/705

(52) U.S. Cl. 435/69.7; 435/320.1; 435/325; 530/395; 536/23.5

(57) ABSTRACT

The invention relates to conjugates comprising all or part of the amino acid sequences of at least one peptide derived from an extracellular loop of the P-170 protein. The peptide may be covalently attached to spacers which may be polyethyleneglycol (PEG), polyglycine, polylysine or any polymer chain suitable for human use and is coupled at its free end to a phospholipids, e.g., phosphatidylethanolamine or any other chemically suitable phospholipid.

This application is a continuation in part application of U.S. Ser. No. 10/902,276, filed Jul. 30, 2004, which is a continuation of International Application No. PCT/EP2004/008330, filed Jul. 25, 2004, which claims the benefit to French application No. FR0309188, filed Jul. 25, 2003. The content of these applications is incorporated herein by reference in their entirety.

### FIELD OF THE INVENTION

The present invention relates to novel agents and compositions useful for treating multidrug resistance (pleiotropic resistance or multidrug resistance) which occurs in certain patients during the treatment of cancers as well as uses thereof.

The publications, patents and other materials used herein to illustrate the invention and, in particular, to provide additional details respecting the practice are incorporated by reference.

### BACKGROUND OF THE INVENTION

The phenomenon of multidrug resistance (MDR) was demonstrated at the end of the 1970s on cancer cell lines that were rendered resistant to chemotherapeutic drugs, in particular drugs used for the treatment of cancers. Multidrug resistance is characterized by a pleiotropy of resistances with respect to chemotherapeutic drugs used for the treatment of a patient, these drugs having different structures and specificities. Among the drugs capable of selecting or inducing pleiotropic resistance of cancer cells, are colchicine, adriamycin, actinomycin, vincristine, vinblastine and mitoxantrone. Phenotypically, multidrug resistance is characterized by a decrease in the intracellular accumulation of cytotoxic drugs, physiological modifications of the cell and overexpression in the cell membrane of P-glycoprotein, also called P-gp protein or alternatively P-170 protein (Van der Bliek et al. 1988. Gene 71(2): 401-411, Thiebaut et al. 1987 Proc. Natl. Acad. Sci. 84(21): 7735-7738, Endicott et al. 1989 Annu. Rev. Biochem. 58: 137-171). The P-170 protein is responsible for an active flux of medicaments out of the cell (also called active efflux), a phenomenon dependent on ATP consumption. The recognition by the P-170 protein, and the P-170 protein-mediated excretion out of the treated cell of a large variety of chemical compounds having diverse structures and functions remains one of the most enigmatic aspects of the function of this protein. The lack of a demonstrated common structural characteristic between the drugs which are the subject of cross resistance is a major hurdle in the development of drugs which would not be excreted under the influence of P-170 protein.

Multidrug resistance of tumors to chemotherapy agents constitutes a central problem in medical cancerology. While progress in support treatments have been made, the problem of drug resistance remains an obstacle to obtaining better cure rates. In fact, tumor cells may not respond to chemotherapy right from the beginning of treatment. This de novo multidrug resistance is unfortunately common in several types of solid tumors. Moreover, the phenomenon of acquired resistance, which manifests itself in tumors which, at the beginning, responded to chemotherapy but which subsequently develop, more or less rapidly, resistance to treatments, has been widely observed.

To enhance efficiency, anticancer treatments have been combined with multidrug resistance-modulating agents, also called reverting agents, which can block the P-170 protein-mediated efflux of drugs out of the cell and thus circumvent multidrug resistance. However, existing reverting agents, such as verapamil, quinine and cyclosporin, result in toxicity that is unacceptable for the patient when used at the doses required for effectively inhibiting the efflux activity of the P-170 protein. For example, verapamil rapidly showed its limits in the treatment of cancer reversion since patients developed dysfunctions such as hypotension, cardiac arrhythmia and congestive heart failure when it was administered- at curative dosages, which are also the limiting doses for toxicity (Miller et al. 1991. J Clin Oncol 9(1): 17-24).

More recent analogues, such as dexverapamil, PSC 833 (cyclosporin derivative) and, most recently, S9788 from Laboratoires Servier, have been the subject of clinical trials that were aimed at overcoming multidrug resistance. However, these novel reverting agents have limits that are comparable to those reported for the prior generation of reverting agents. In fact, the trials for treatment of multidrug resistance using S9788 (6-[4-[2,2-di-(4-fluorophenyl)ethylamino]-1-piperidinyl]-N,N′-di-2-propenyl-1,3,5-triazine-2,4-diamine), a triazineaminopiperidine derivative, have characterized the limits for use of this product, subsequent to the appearance of phenomena of cardiac toxicity, ventricular arrhythmia and. torsade de pointe (Stupp et al. 1998. Ann Oncol 9(11): 1233-1242). Consequently, the multidrug resistance phenomenon is difficult to tackle with reverting agents, novel and conventional, since treatment doses turned out to be equivalent to the thresholds of toxicity for the patient who has become refractory to chemotherapy.

Immunotherapy, in particular the use of monoclonal antibodies, has also been considered for treating multidrug resistance in a patient. It was tested first for inhibiting the formation of tumors in ovarian cancer using the monoclonal antibody MRK16 (Tsuruo. 1989. Cancer Treat Res 48: 1811-1816). More recently, monoclonal immunotherapy for the treatment of multidrug resistance has been investigated more thoroughly by Mechetner and Roninson (1992. Proc Natl Acad Sci USA vol. 89 pp. 5824-5828). In fact, monoclonal antibodies UIC2 directed against an extracellular epitope of human P-glycoprotein were obtained and tested in vitro on cell lines resistant to anticancer agents. It was shown in vitro that the inhibitory effect of the monoclonal antibodies UIC2 is comparable to that of verapamil used at maximum clinical doses (3 μM). The anti-P-170 monoclonal antibodies exert their effect by inhibiting (a) the ATPase activity of the P-170 protein and (b) the binding of medicinal products to the P-170 protein.

An appropriate immunotherapy, based on the injection of monoclonal antibodies into a patient, can have certain advantages since it can eliminate residual resistant cells of a tumor. However, the lack of knowledge of the specificity, toxicity, efficacy and of the mechanism of action of the antibodies limits the use of this approach for overcoming multidrug resistances due to the overexpression of the P-170 protein.

### SUMMARY OF THE INVENTION

In one embodiment, the present invention provides an alternative strategy to available treatments for multidrug resistance in cancer, remedying, at least partly, the disadvantages of known treatments for multidrug resistance. The invention is also directed to an immunotherapy based on the induction of polyclonal auto-antibodies specific for P-glycoprotein (P-170 protein). This immunotherapy uses, in certain embodiments, the antigenic capacity of conjugates comprising peptides derived from at least one of the extracellular loops of the. P-170 protein to induce antibodies in a patient when these peptides are presented and/or administered in a form which allows or promotes the expression of the antigenic capacity. In particular, the antibodies are auto-antibodies against human P-glycoprotein.

The invention relates also to an immunogenic composition comprising as antigenic structure, conjugates comprising all or part of the amino acid sequences of at least one peptide derived from an extracellular loop of the P-glycoprotein, each peptide being combined with at least two molecules of fatty acid containing a carbon chain of between C12 and C24, so as to allow, under suitable administration conditions, the induction of anti-P-170 antibodies.

The invention is also directed to a conjugate comprising at least one peptide based on an extracellular loop of a P-glycoprotein, the peptide having a N- and C-terminal end,
one or more terminal amino acids attached to said N- and C-terminal end of the peptide,
at least two spacer molecules each having two termini,
a first of the termini being covalently attached to a phospholipid, and
a second of the termini being covalently attached to one of the terminal amino acids.

In certain embodiments, the invention is directed to a conjugate comprising
at least one peptide derived from an extracellular loop of a P-glycoprotein, the peptide having a N- and C-terminal end,
one or more terminal amino acids attached to the N- and C-terminal ends of the peptide,
at least two spacer molecules each having two termini
a first of the termini being covalently attached to a phospholipid, and
a second of the termini being covalently attached to one of the terminal amino acids.

In certain embodiments, the invention is directed at agents which can be used to induce an immune response in patients suffering from multidrug resistance in the course of a cancer treatment and may in certain embodiments reduce or even substantially reverse this resistance. The invention also relates to agents for preventing the appearance of multidrug resistance.

The invention also relates to methods of immunization using the above compositions or being based on the above conjugates. The invention relates also to methods of immunization which precedes, is concomitant with, or follows a chemotherapeutic treatment administered to a patient.

The invention also relates to the use of the composition according to the invention, for the in vivo treatment of multidrug resistance in a patient suffering from a cancer treated with anticancer medicaments or, where appropriate, for the prevention of such a multidrug resistance.

The invention is also directed towards methods for preparing the conjugates of the present invention.

One of these methods comprises synthesizing an amino acid sequence comprising at least one peptide derived from an extracellular loop of a P-glycoprotein and one or more terminal amino acid(s) on a solid support. At least one of said terminal amino acid(s) may be side chain protected. The side chains are deprotect and the amino acid sequence are released from the solid support by cleavage with a mild acid. A pre-activated spacer-phospholipid molecule may be coupled to at least one of the deprotected terminal amino acid(s), and the resulting conjugate may, optionally, be purified.

In a related method the peptide does not contain internal attachment points for spacer-phospholipid molecules. Here the synthesized amino acid sequence is released from the solid support by cleavage with a harsh acid, after deprotecting the side chains of terminal amino acid(s). A pre-activated spacer-phospholipid molecule is coupled to the deprotected terminal amino acid(s) and the resulting conjugate is optionally purified.

The invention is also directed to a hybridoma which produces a monoclonal antibody specifically immunoreactive with the conjugates of the present invention and monoclonal antibodies produced by such a hybridoma. The invention is also directed to an immunological assay for detecting P-glycoprotein antigen in a biological sample and a method for the immunological detection of cancer via such antibodies.

The invention is also directed to a method for producing an antibody comprising providing a conjugate of the present invention, raising antibodies in vitro or in vivo against at least one peptide of the conjugate, and optionally, isolating said antibodies.

The invention is furthermore directed to an immunological assay for detecting P-glycoprotein antigen in a biological sample comprising
providing a conjugate of the present invention, producing a monoclonal antibody specifically immunoreactive with a peptide of the conjugate,
combining the monoclonal antibody with the biological sample; and assaying the biological sample for antigen binding as a measure of a monoclonal antibody-P-glycoprotein antigen complex formed in said combining step.

Surprisingly and unexpectedly, a 77% increase in survival time in mice immunized with immunogenic compositions and after inoculation of the cancer cells, followed by a chemotherapeutic treatment plane, was observed.

These results are very promising in particular in view of other published results obtained when treating multidrug resistance in the same cancer model with another substance. The respective publications described as the maximum a 49% increase in survival of mice treated with such another substance (Pierré et al. 1992. Invest New Drug. 10: 137-148). In addition, Yang et al. (1999. BBRC. 266: 167-173) observed, with the same cell line, only a 35% increase in the survival of mice treated with vincristine and cyclosporin A.

### BRIEF DESCRIPTION OF THE DRAWINGS

The present invention is illustrated, without however being limited, by the following figures:

FIG. 1: Representation of the synthetic peptides corresponding to the extracellular fragments of the mouse P-170 protein, coupled to four molecules of palmitic acid (C16) per molecule of peptide.

FIG. 2: Diagrammatic representation of the Boc/benzyl strategy for synthesizing the peptides on a solid support.

FIG. 3: Protocol for chemotherapy after immunization and injection of P388R cells in mice.

FIG. 4: Representation of the antibody titer as a function of the immunization time (1st, 2nd, 3rd injection) in the sera of mice immunized with Lp2. The anti-mpp1, mpp2 and mpp4 antibodies were quantified and the various isotypes were detected using Ig (M, G3, G2a, G2b, G1) specific anti-murine secondary antibodies, respectively. Each histogram represents the mean of the values obtained for 5 sera of mice bled 12 days after an immunization. One unit corresponds to 0.2 μg Ig/ml.

FIG. 5: Representation of the antibody titer as a function of the immunization time (1st, 2nd, 3rd injection) in the sera of mice immunized with Lp4. The anti-mpp1, mpp2 and mpp4 antibodies were quantified and the various isotypes were detected using Ig (M, G3, G2a, G2b, G1) specific anti-murine secondary antibodies, respectively. Each histogram represents the mean of the values obtained for 5 sera of mice bled 12 days after an immunization. One unit corresponds to 0.2 μg Ig/ml.

FIG. 6: Representation of the antibody titer as a function of the immunization time (1st, 2nd, 3rd injection) in the sera of mice immunized with Lp3. The anti-mpp1, mpp2 and mpp4 antibodies were quantified and the various isotypes were detected using Ig (M, G3, G2a, G2b, G1) specific anti-murine secondary antibodies, respectively. Each histogram represents the mean of the values obtained for 5 sera of mice bled 12 days after an immunization. One unit corresponds to 0.2 μg Ig/ml.

FIG. 7:. Representation of the antibody titer as a function of the immunization time (1st, 2nd, 3rd injection) in the sera of mice immunized with Lp1. The anti-mpp1, mpp2 and mpp4 antibodies were quantified and the various isotypes were detected using Ig (M, G3, G2a, G2b, G1) specific anti-murine secondary antibodies, respectively. Each histogram represents the mean of the values obtained for 5 sera of mice bled 12 days after an immunization. One unit corresponds to 0.2 μg Ig/ml.

FIG. 8: survival time of the mice immunized with Lp1 and Lp2. At time 0, 10⁶ chemoresistant P388R cells were inoculated. On days 1, 10 and 22, 5.5 mg/kg of doxorubicin were injected and on days 4 and 14, 2.5 mg/kg of vinblastine were injected.

FIG. 9: Synthesis of conjugates in which amino acid sequences and phospholipids are attached to each other via PEG.

### VARIOUS AND PREFERRED EMBODIMENTS OF THE INVENTION

P-glycoprotein or “P-170 protein” is a 170 kDa membrane-bound phosphoglycoprotein which was identified by Juliano and Ling (1976. Biochim Biophys Acta 455(1): 152-162). The murine P-170 protein consists of 1276 amino acids forming two equivalent entities. The hydrophobic domains of the molecule are numbered from 1 to 12 and are involved in the efflux of chemotherapeutic drugs. Extracellular loops 1, 2 and 4 of the murine P-170 protein have been selected for their pronounced extracellular topology, suggesting that they may be antigenic in nature. The human P-170 protein (Chen et al. 1986 Cell. 47(3): 381-389) is a 1280 amino acid protein having two homologous domains each comprising six transmembrane helical domains and a nucleotide binding site. The hydrophobic regions of these transmembrane domains form extracellular loops considered to be the fragments for recognition of the P-170 protein from the outside of the cell. Extracellular loops 1, 4 and 6 of the human P-170 protein have also been selected as having a high antigenic capacity due to their particularly marked extracellular locations.

The immunotherapy of the present invention allows the effects of the chemotherapeutic drugs used in cancer chemotherapy to manifest themselves.

In the context of the invention, “cancer” is-defined, in accordance with its usual meaning, by two main characteristics: cell growth and proliferation that is not regulated by external signals, and the ability to invade tissues along with, where appropriate, the ability to form metastases by colonizing distant sites.

These characteristics are the result of the intrinsic properties of cancer cells, i.e., of their caryotypic and genomic instability, of their uncontrolled proliferation, of their metastatic capacity accompanied by the acquisition of new phenotypes and also of the activation and derepression of oncogenes in said cancer cells. The term “cancer” is therefore, in the context of the present invention, any phase of cell growth or proliferation having the above characteristics, evolving in particular toward the development of primary tumors and/or of metastatic tumors (secondary tumors).

In addition, for the purposes of the present invention, the expression “treatment for multidrug resistance” is understood to mean all medical treatments intended to combat multidrug resistance, to limit the consequences thereof, to avoid death and/or, preferably, to re-establish sensitivity to anticancer medicinal products. Towards this goal, the treatment is preferably directed toward curing the multidrug resistance, by inducing complete reversion to a chemotherapy-sensitive cell phenotype. This reversion may, however, be partial: consequently, the treatment of multidrug resistance will prove to be palliative, allowing prolonged remission of the patient. The treatment for multidrug resistance is also characterized by virtue of its prophylactic capacity, making it possible to prevent the appearance of de novo or acquired multidrug resistances in a patient. That means, a treatment for multidrug resistance according to the present invention includes a vaccination for the same.

The invention relates, in certain embodiments, to an immunogenic composition comprising conjugates comprising all or, in certain embodiments, part of the amino acid sequences of an extracellular loop of the P-170 protein that, under suitable administration conditions, allows the induction of anti-P-170 antibodies.

In the context of the present invention, the immunogenic composition also allows the induction of anti-P-170 antibodies for the treatment including the partial or full reversion of multidrug resistance as well as the prophylaxis against multidrug resistance in a patient suffering from a cancer.

In preferred embodiments, the conjugates comprise part, or preferably, all of the amino acid sequences of at least one extracellular loop, two extracellular loops, preferably of at least three extracellular loops, of the P-170 protein.

A “conjugate,” according to the invention, is a reagent formed by covalent bonding between molecules of fatty acids containing a carbon chain of between C12 and C24, and peptide according to the present invention, to form a lipid-peptide hybrid molecule. The peptide, which is, in a preferred embodiment obtained, by solid-phase synthesis, is covalently coupled to the fatty acid molecules constituting the lipid region of the molecule. Alternatively, the peptide may be covalently coupled to at least two spacer molecules, including, but not limited to polyethyleneglycol (PEG), polylysine or polyglycine and other suitable polymers, which, in turn are covalently attached, to a phospholipid including, but not limited to, phosphatidylethanolamine. As the person skilled in the art will appreciate, any other chemically suitable phospholipid can be used that allows reconstitution in liposomes and virosomes including their functional equivalents.

For the purposes of the present invention, the term “derived peptide” or “peptide derived” from a P-glycoprotein or loops thereof denotes all or part of the amino acid sequence making up an extracellular loop of the P-glycoprotein, as long as said “derived peptide” has at least one epitope of the extracellular loop from which it derives. The inventions also includes peptides based on the P-glycoprotein or loops thereof that are peptides comprising all or part of the amino acid sequence making up, for example, an extracellular loop of the P-glycoprotein as long as these peptides, under appropriate administration conditions, alone or with other such peptides, induce an antibody response. Such peptides advantageously have between 5 and 50 amino acid residues, preferably between 5 and 40, or between 10 and 30, advantageously between 10 and 25 residues. The following falls within this definition: (1) peptides whose amino acid sequence is identical to the corresponding sequence of the extracellular loop from which they are derived, or (2) peptides whose amino acid sequence is modified with respect to the sequence of the extracellular loop from which they are derived, wherein said modification may, in certain embodiments, consist of a point mutation by insertion, deletion or substitution, in particular, conservative substitution, of one or more residues, provided that the peptide formed thereby still carries an epitope of the P-glycoprotein or induces an antibody response as outlined above, respectively. In particular, an acceptable mutation is a mutation which does not disturb the conformation of the modified peptide when it is included in a composition of the invention. This can be verified by the ability of the modified peptide to induce antibodies when it is formulated in a composition in accordance with the invention.

The present invention also includes peptides that have substantial homology or substantial identity with the respective portion of the naturally occurring P-glycoprotein. The terms “substantial homology” or “substantial identity”, when referring to a peptide of the present invention, indicate that the peptide in question exhibits at least about 30% identity with the respective portion of the naturally-occurring protein, usually at least about 70% identity, more usually at least about 80% identity, preferably at least about 90% identity, and more preferably at least about 95% identity.

Homology, for polypeptides, is typically measured using sequence analysis software. See, e.g., the Sequence Analysis Software Package of the Genetics Computer Group, University of Wisconsin Biotechnology Center, 910 University Avenue, Madison, Wis. 53705. Protein analysis software matches similar sequences using measures of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

The peptides of the invention are preferably obtained by chemical synthesis, in particular using the methods described hereinafter.

In the context of the invention, the expression “extracellular loop” of P-glycoprotein (or P-170 protein) denotes each amino acid sequence of the P-170 protein having an extracellular topology or a significant connection with the extracellular environment so that it can be selected as a sequence carrying an epitope of this protein.

The amino acid sequences making up the synthetic peptides used in the context of the invention may be antigenic per se, or may be antigenic when they are presented in a conformation that conserves the conformation of the amino acid sequence of the extracellular loop from which the peptide is derived, or a conformation sufficiently similar to confer on the peptide the ability to induce the production of the same or functionally equivalent antibodies under suitable administration conditions. The ability to induce the production of antibodies may, for example, be verified in mice immunized with the peptides prepared in according to the invention or by any other known means. Thus, the peptides of the invention advantageously have, in the immunogenic composition, a three-dimensional conformation that reproduces the conformation of the extracellular portion of loop from which the peptides are derived, or are so similar to said conformation, as to confer on the composition formed their immunogenic capacity. The appropriate presentation of the peptides advantageously results from their association with the other constituents of the immunogenic composition.

This ability to induce the production of antibodies is in particular present when the peptides of the invention are peptides that have been synthesized and modified to be in the form of conjugates and are combined with liposomes or virosomes including their functional equivalents.

In the context of the invention, the expression “antigenic structure” denotes molecules capable of reacting with antibodies so as to form antigen/antibody complexes. An “antigenic structure” of the immunogenic composition may or may not have, as such, the ability to induce a phenomenon of immunogenicity corresponding to the formation of antibodies specific for a given antigen. An immunogenic composition according to the present invention, comprises in a preferred embodiment, liposomes. In another preferred embodiment, in particular in the context of conjugates containing spacer-phospholipid molecules, the conjugates are combined with an adjuvant, such as lipid A, in particular, monophosphoryl lipid A (MPLA). Particularly preferred are compositions comprising conjugates, liposomes and further comprising an adjuvant such as lipid A, in particular monophosphoryl lipid A (MPLA).

Peptides derived from an extracellular loop of the P-170 protein of any species, for example mouse and human, are part of the present invention. Representative extracellular loops of the murine P-170 protein, the full sequence of which is as of December 2004 disclosed under Swiss-Prot accession number P06795 and is incorporated herein by reference in its entirety, are:
loop 1 (SEQ ID 1): GNMTDSFTKAEASILPSITNQSGPNSTLIISNSSLEEE;
loop 2 (SEQ ID 2): KVLTSFTNKELQAYAK; and
loop 4 (SEQ ID 3): SRDDDHETKRQNCN.

Suitable subsequences of loop 1 are shown in large letters in Table III.

Murine peptides derived from loops 1, 2 or 4 are referred to herein as murine peptide 1, 2 and 4, respectively. The amino acid sequence of these peptides may be extended, in particular at their C and N terminal end, by one or more amino acid residues, including but not limited to, lysine. Amino acid residues that extend the amino acid sequence of a derived peptide are referred herein as “terminal” residues or “terminal amino acid residues.” An “end-standing residue” or “end-standing amino acid residue” is the “terminal” amino acid residue that is located at the very end of an amino acid sequence. In certain embodiments, two lysines (terminal lysines) are added at both ends of the peptide, in other embodiments, a combination of glycines and lysines are added at each end. Those lysines and glycines may act as attachment points for fatty acid molecules or activated spacer-phospholipid molecules to form conjugates according to the present invention.

By way of examples, conjugates according to the present invention such, as murine palmitotylpeptides (mpp), comprise peptides derived from extracellular loop 1 (mpp1), from at least one of extracellular loops 2 (mpp2) or 4 (mpp4) of the murine P-170 protein, which were characterized by their sequence, their molecular mass and their purity after synthesis according to the experimental protocol as shown below in Table I. The immunogenic composition of the invention may comprise a combination of conjugates comprising peptides derived from different loops.

TABLE I

| | |
|---|---|
| Calculated | Observed |

| | | | |
|---|---|---|---|
| Conjugate | molecular | molecular | Purity |

| | | | | |
|---|---|---|---|---|
| name | Amino acid sequence | mass | mass | (%) |

| | | | | |
|---|---|---|---|---|
| Mpp1 SEQ | [symbol] GNMTDSFTKAEASILPSITNQ | 5436 | 5437 | 91.6 |
| ID NO 4 | SGPNSTLIISNSSLEEE [symbol] | | | |
| Mpp2 SEQ | [symbol] KVLTSFTNKELQAYAK [symbol] | 3293 | 3293 | 93.8 |
| ID NO 5 | [symbol] | | | |
| Mpp4 SEQ | [symbol] SRDDDHETKRQNCN [symbol] | 3190 | 3188 | 95.3 |
| ID NO 6 | [symbol] | | | |

The calculated and observed molecular masses of the peptides derived from extracellular loops 1, 2 and 4 of the murine P-170 protein were obtained by analysis by mass spectrometry, namely:
[M+H+] for the calculated molecular mass,
MALDI-TOF and PDMS-TOF for the measured molecular mass, i.e. (Matrix Assisted Laser Desorption Ionization—Time of Flight) (Plasma Desorption Mass Spectrometry—Time of Flight).

In addition, the amino acid sequences were also analyzed by hydrolysis of aliquots in 6N HCl/phenol at 110° C. in order to verify the expected and obtained number of amino acids (expected/obtained):
mpp 1, A (2/1.5), D (5/4.4), E (5/6.6), F (1/0.6), G (4/3.4), I (4/3.6), K (4/4.0), L(3/3.5), M (1/0.7), P (2/1.8), T (4/3.0), S (8/7.4).
mpp 2, A (2/2.3), D (1/1.2), E (2/2.4), F (1/1.0), G (2/2.1), K (6/6.0), L (2/2.1), S (1/0.9), V (1/1.0), Y (1/1.0).
mpp 4, D (5/5:0), E (3/3.1), G (2/2.0), K (4/3.7), S (1/0.9), R (2/1.9).

According to a particularly advantageous embodiment of the invention, the immunogenic composition of the present invention comprises conjugates comprising peptides derived from extracellular loop 1 and, from extracellular loops 4 and 6 of the human P-170 protein and combinations thereof. The conjugates of the immunogenic compositions may therefore comprise amino acid sequences derived from extracellular loops 1 and 4 or from extracellular loops 1 and 6.

Preferably, the conjugates of the immunogenic composition comprise peptides derived from the three loops 1, 4 and 6 of the human P-170 protein.

Given the 47-amino acid length of loop 1 of the P-170 protein, peptides derived from this loop may result from loop 1 being divided up into three fragments, obtained by cleavage at the glycosylation sites. The three derived peptides give rise to three peptides 1a, 1b and 1c, corresponding to sub loops 1a, 1b and 1c which can be synthesized. Other derived peptides according to the present invention may-be fragments of any of these three peptides, containing one or more epitopes. Consequently, the conjugates according to the invention comprise all or part of the peptides derived from extracellular loop 1 of the human P-glycoprotein corresponding to the three peptides resulting from cleavage of said loop 1 at the glycosylation sites.

The peptide sequence of the conjugates can be used as they are for preparing immunogenic compositions according to the invention, in particular, in combination with liposomes. The peptides of these conjugates may be chosen, respectively, from the following amino acid sequences, which stem from human P-170 protein as disclosed by Swiss-Prot accession number P08183 as of December 2004, which is incorporated herein by reference:
for loop 1 (SEQ ID NO 7): GEMTDIFANAGNLEDLMSNITNRSDINDTGFFMNLE EDMTRYAYYYS
for loop 1a (SEQ ID NO 8): GEMTDIFANAGNLEDLMS
for loop 1b (SEQ ID NO 9): NITNRSDINDTGFF
for loop 1c (SEQ ID NO 10): MNLEEDMTRYAYYYS
for loop 4 (SEQ ID NO 11): FSKIIGVFTRIDDPETKRQNSNLFS
for loop 4a (SEQ ID NO 12): FTRIDDPETKRQNSNLFS
for loop 6 (SEQ ID NO 13): FRFGAYLVAHKLMSFED and/or a combination thereof.

For extracellular loop 1, the three peptides 1a, 1b and 1c are advantageously used in the same composition. Alternatively, combinations of peptides 1a and 1b or 1a and 1c or 1b and 1c may be used.

The amino acid sequence of the human peptides may also be extended, in particular at their C and N terminal ends, by one or more amino acid residues, including, but not limited to, lysine or glycine or combinations thereof. In certain embodiments, one or two lysines are added at both ends of the peptide, in other embodiments a combination of glycines and lysines are added at each end. Those terminal lysines and glycines may act as attachment points for fatty acid molecules or activated spacer-phospholipid molecules to form conjugates according to the present invention.

Table II recapitulates the amino acid sequences of the conjugates corresponding to extracellular loops 1, 4 and 6 of the human P-170 protein. The amino acid sequences corresponding to the sequences of the derived peptides are in large letters, while the small letters correspond to the added amino acids to which the fatty acid molecules may be coupled.

The sequence, the molecular mass and the purity of the peptides described in Table II can be controlled by means of the techniques described above for the peptides of Table I.

A peptide derived from an extracellular loop advantageously has a purity equal to or greater than 90%, advantageously between 91% and 98%, measured by HPLC chromatography after synthesis.

TABLE II

Amino acid sequence from human P-170

| | |
|---|---|
| Conjugate | human including added terminal amino |

| | |
|---|---|
| name | acids |

| | |
|---|---|
| Hpp1 (SEQ | [symbol] GEMTDIFANAGNLEDLMSNITNRSDINDTGFFMNLE |
| ID NO 14) | EDMTRYAYYYS [symbol] |
| Hpp1a | [symbol] GEMTDIFANAGNLEDLMS [symbol] |
| (SEQ ID | |
| NO 15) | |
| Hpp1b | [symbol] NITNRSDINDTGFF [symbol] |
| (SEQ ID | |
| NO 16) | |
| Hpp1c | [symbol] MNLEEDMTRYAYYYS [symbol] |
| (SEQ ID | |
| NO 17) | |
| Hpp4 (SEQ | [symbol] FSKIIGVFTRIDDPETKRQNSNLFS [symbol] |
| ID NO 18) | |
| Hpp4a | [symbol] FTRIDDPETKRQNSNLFS [symbol] |
| (SEQ ID | |
| NO 19) | |
| Hpp6 (SEQ | [symbol] FRFGAYLVAHKLMSFED [symbol] |
| ID NO 20) | |

In the context of the present invention, the amino acid sequences corresponding to the sequences of the extracellular loops (represented in large letters) can be extended, in particular at their end(s), with amino acid residues (represented in the peptides illustrated herein in small uppercase letters) to which, for example, the fatty acid residues are coupled. Advantageously, the molecules of fatty acid contain a C12, C13, C14, C15, C16, C17, C18, C19, C20, C21, C22, C23 or C24 carbon chain are preferably C16 palmitic acid molecules. The carbon chains of the fatty acid molecules of between C12 and C24 are linear or branched. Preferably, the fatty acid molecules have linear carbon chains. To avoid reaction incompatibility during the peptide synthesis, in particular during the final step of deprotection in the presence of strong acid, the fatty acid molecules of the present invention are, in certain embodiments, neither mono- nor polyunsaturated.

Preferably, each conjugate comprises at least four molecules of fatty acid containing a carbon chain of between C12 and C24, the fatty acid molecules are preferably distributed at the N- and C-terminal ends of the peptides. However, other distributions of the fatty acid molecules are within the scope of the invention such as within the amino acid sequence. The peptides of conjugates may be each coupled to four molecules of palmitic acid; they are therefore tetrapalmitoylated.

Preferably, two molecules of palmitic acid are coupled to the N-terminal end and two molecules of palmitic acid are coupled to the C-terminal end of the peptide.

Preferably, and as illustrated in Table II, the amino acid sequences of the extracellular loops of the peptides of the conjugates are extended in the N- and/or C-terminal position by one or more amino acid residues, to allow combination with the molecules of fatty acid containing a carbon chain of between C12 and C24 or with spacer-phospholipid molecules. The phospholipid moiety of the spacer-phospholipid molecule is preferably phosphatidylethanolamine, wherein the fatty acid moiety of the phosphatidylethanolamine is preferably as described above.

The peptides may be combined with said fatty acids or spacer-phospholipid molecules exclusively in the N-terminal position or, alternatively, exclusively in the C-terminal position. Advantageously, in the conjugates, the combining of the peptides with the fatty acids or spacer-phospholipid molecules is carried out in the N-terminal and C-terminal position of the peptide sequences, in particular so as to give tetrapalmitoylated sequences.

Alternatively or cumulatively, fatty acids or spacer-phospholipid molecules may be attached to internal residues in the peptide sequence.

“Pegylation”, i.e. the process comprising covalently coupling a PEG molecule to a peptide, to increase the immunogenicity/immunogenic nature of a peptide used as antigen, is a technique well known to those skilled in the art (2002. Adv Drug Deliv Rev. 54(4): 459-476).

This process makes it possible in particular to increase the accessibility of the peptide sequence and, in this way, the presentation of the antigen.

In general, with longer peptide sequences, a greater number of molecules of PEG are preferred.

Advantageously, molecule(s) of PEG, from 2 to 8000, preferably PEG having between about 50 and 100 units, is(are) attached to, for example, a lysine (K) residues found in the N- and/or C-terminal position of the amino acid sequences of the extracellular loops of the peptides of the conjugates of the present invention. Each peptide may be covalently attached to at least two molecules of fatty acid containing a carbon chain of between C12 and C24 as described above or to at least two spacer molecules, such as two chains of polyethylene glycol (2-8000) which are each coupled to a phospholipid such as phbsphatidylethanolamine to make it possible to reconstitute these antigenic complexes in the lipid bilayer of, e.g., the liposomes. Under suitable administration conditions, these complexes reconstitute, in adjuvant-liposomes, leading to the induction of anti-P-170 antibodies.

A “spacer” according to the present invention is a molecule that can form a bridge between an amino acid residue, such as a terminal lysine or an analogue thereof, and a phospholipid, such as phosphatidylethanolamine, in the conjugates of the present invention. Those molecules include, but are not limited to, PEG having 2 to 8000 units, polyglycine having preferably between 2 and 100 residues and polylysine molecules having preferably between 2 and 100 residues. A spacer molecule may be attached to a phospholipid of the present invention to form a spacer-phospholipid molecule. However any other suitable polymers may be used as spacers. The coupling of one or more peptides according to the present invention to one or more of those spacer-phospholipid molecules results in a conjugate according to the present invention. The peptide may be attached to the spacer-phospholipid molecule via the ε amino group of one of the one or more lysine(s) that have been added to the peptides of the present invention.

To form the immunogenic compositions of the invention from the abovementioned conjugates, liposomes are advantageously selected as carrier for presenting the conjugates in the composition of the present invention.

Advantageously, said conjugates are present at the surface of the liposomes.

For the purposes of the present invention, the term “liposome(s)” is understood to mean an artificial spherical particle comprising or consisting of one or more layers of phospholipids.

The composition according to the present invention advantageously comprises the conjugates and the liposomes in a molar ratio of between 1/10 and 1/1000, preferably between 1/50 and 1/500, advantageously in a molar ratio of 1/250.

Advantageously, the liposomes are prepared by mixing dimyristoylphosphatidylcholine (DPMC), dimyristoylphosphatidylglycerol (DMPG) and cholesterol in a molar ratio of, respectively, 0.9:0.1:0.7. The products used above are preferably of synthetic origin in order to avoid the possibilities of contamination with endotoxins, prions or viruses. For example, the DMPC and DMPG phospholipids are of synthetic origin (Avanti Polar Lipids USA) and the cholesterol, which is 98% pure, is of animal origin. Monophosphoryl lipid A (MPLA), also of synthetic origin, and known to increase the immune response (Fries et al. 1992. Proc Natl Acad Sci 89(1): 358-362) was added to liposomes, and tested, at a concentration of 40 μg per pmol of phospholipids.

Thus, the composition according to the present invention may also comprise alum and/or immunomodulators such as, but not limited to, lipopolysaccharide (LPS) derivative such as lipid A, monophosphoryl lipid A (MPLA) or a synthetic lipid A analogues and/or microcapsules of proteins and of polysaccharides and/or muramyldipeptide (MDP) and muramyltripeptide (MTP) and derivatives thereof. Immunostimulating agents have been reviewed by Werner & Jolles (Werner & Jolles, Eur. J. Biochem. 242, 1-9 (1996)). Such immunogenic compositions are generally prepared in the form of liquid solutions, or of injectable suspensions, or else in a solid form suitable for solubilization prior to injection in the context, for example, of a kit for making use of the present composition, as described below.

The invention also relates to peptides which are derived from at-least one extracellular loop of the P-170 protein and which cause the induction of anti-P-170 antibodies when they are used in the immunogenic compositions as described. These peptides may, respectively, be chosen from the following amino acid sequences:

SEQ ID NO 1, SEQ ID NO 2, SEQ ID NO 3, SEQ ID NO 7, SEQ ID NO 8, SEQ ID NO 9, SEQ ID NO 10, SEQ ID NO 11, SEQ ID NO 12, and SEQ ID NO 13.

Also included in the present invention are the nucleic acid, DNA or RNA, sequences encoding the peptides as described above. Reference is made hereby to U.S. Pat. No. 5,198,344 to Croop et al and U.S. Pat. No. 5,206,352 to Roninson et al which are incorporated herein by reference in their entirety.

The present invention relates not only to the immunogenic composition and conjugates per se and as defined above, but also to methods for preparing the conjugates and composition.

The experimental results below show that the combining of specific peptides with only two molecules of palmitic acid may not result in the expression of an immunogenic response. This reflects the need to select the fatty acids so as to confer on the conjugates formed, the conformation required for the induction of an immune response.

Particular attention is also given to the synthesis of the conjugates according to the invention, and in particular to their peptide region. In fact, the previous studies by Tosi et al. (1995) demonstrated that the peptide sequence of mppl, which is the longest of the conjugates, with 43 amino acids, did not bring about an immune response. In the absence of explanations or working hypotheses for this observation, the inventors have revealed, in the context of the present invention, the essential role of a very precise peptide synthesis. The inventors have therefore developed and shown that an improved method of synthesis allowing a more effective production of certain amino acids by avoiding their involvement in early terminating reactions makes it possible to obtain in particular an immunoreactive conjugates. In one embodiment of the invention, conjugates can therefore be obtained in a suitable form for producing the immunogenic composition of the invention by means of a solid-support synthesis according to the Boc/benzyl strategy.

In the case in point, as described in the example section, the peptides corresponding to the sequences of extracellular loops 1, 2 and 4 of murine P-170 were therefore synthesized via an Applied Biosystems 430A peptide -synthesizer using the tert-butyloxycarbonyl/benzyl or Boc/benzyl strategy and, in situ, activation with (N-[(1H-benzotriazol-1-yl)(dimethylamino)methylene]-N-methylmethanaminium hexafluorophosphate N-oxide.

The synthesis of the peptides can be carried out on a peptide synthesizer, for example an Applied Biosystem 430A synthesizer, using (13,14)-tert-butyloxycarbonyl/benzyl and in situ activation with (N-[(1H-benzotriazol-1-yl)(dimethylamino)methylene]-N-methylmethanaminium hexafluorophosphate N-oxide (Schölzer M. et al Science 1992, 256 (5054): 221-225).

According to this method, the synthesis of the conjugates is based on the synthesis of the amino acid sequences of the extracellular loops of the peptides of the conjugates on a solid support according to the Boc/benzyl strategy one or more amino acid residues are introduced at the N and/or C-terminal end so as to allow combination with the molecules of the fatty acid containing a carbon chain of between C12 and C24, followed by a step consisting of deprotection of the amine functions of the N and C-terminal lysines in order to couple them with the fatty acids containing a carbon chain of between C12 and C24. The final step is cleavage with a strong acid, such as anhydrous hydrofluoric acid, of the conjugates synthesized on the solid support, such as a resin.

This improved method of synthesis allows a more effective production of certain amino acids by avoiding their involvement in early terminating reactions.

The combining of said peptides with the fatty acids can be carried out either in the N-terminal position or, alternatively, in the C-terminal position.

Advantageously, the combining of the peptides with the fatty acids is carried out in the N-terminal and C-terminal position of the peptide sequences, in particular to produce tetrapalmitoylated sequences.

Alternatively or cumulatively, it is possible to combine the fatty acids with internal residues in the peptide sequence.

Optionally, once they have been synthesized, the conjugates are purified, for example by RP-HPLC or reverse-phase high performance liquid chromatography. This purification step is made delicate by the presence of a lipid chain, which results in a broadening of the chromatographic peaks and solubility problems. Isolation of the expected product, from the impurities formed, during the elongation of the peptide, can be difficult and can result in low yields. In addition, the purification may be difficult insofar as the fatty acid is located in the C-terminal position. In fact, in this case, the desired product, but also the impurities, carry the lipophilic portion responsible for the chromatographic difficulties. In addition, since this strategy involves, at the end of synthesis, a step of cleavage and of deprotection in strong medium, this treatment limits the choice of the lipophilic portion (Deprez et al. 1996. Vaccine 14(5): 375-382; Stöber et al. (1997) Bioorg. Med. Chem. 5(1): 75-83).

Other strategies that result in a very precise peptide synthesis can also be used. The Fmoc strategy is, in particular, of advantage in the context of the synthesis of conjugates containing spacer-phospholipids. Here, partially side chain protected peptides may be produced, that is, peptides having unprotected terminal amino acids. The production of such partially side chain protected peptides is, in particular, preferred if the peptide contains internal amino acids, such a lysine or histidine, that might react with preactivated non-peptidic moieties which are discussed below. After resin cleavage, those partially side chain protected peptides may be coupled to preferably at least two non-peptidic moieties, that are preferably preactivated, such as preactivated spacer-phospholipid molecules, to form a conjugate according to the present invention. This coupling takes place preferably in solution with at least a solvent and, preferably, in the presence of an excess of amine base. According to this method, the synthesis of a epitope carrying sequence comprising the amino acid sequence of an extracellular loop of a peptide derived from a P-170, molecule on a solid support is advantageously based on orthogonal Fmoc/tert-butyl chemistry. The N- and/or C-terminal position of the sequence contains one or more amino acid residue, preferably lysine residue, more preferably, two lysine residues, that allow site specific conjugation of a, preferably pre-activated, spacer-phospholipid molecule, by, for example, an amide linkage. If internal lysine and/or histidine residues are present, those terminal lysine residues may be protected with an orthogonal butyl, such as 1-(4,4-Dimethyl-2,6-dicyclohexylidene) 3-methyl-butyl (ivDde). In a preferred embodiment of this method, removal of the N-terminal Fmoc group, and optional acetylation of the free amino terminal group, is followed by chemoselective deprotection of the orthogonal butyl and selective resin cleavage. The release of the partially side protected peptides having unprotected terminal amino acid residues is followed by the coupling of at least one unprotected terminal amino acids, preferably at each end, to a pre-activated form of a spacer-phospholipid molecule. Only after the coupling reaction is quenched, the remaining side chain protecting groups are removed with, for example, a standard acidic cocktail. If no internal lysine and/or histidine residues are present, tert-butoxycarbonyl (Boc) groups can be used to protect the terminal amino acids. In this case, deprotection and resin cleavage can be performed simultaneously and is followed by coupling the fully deprotected sequence via its terminal amino acids to a pre-activated form of a spacer-phospholipid molecule.

An “activated” or “pre-activated” spacer-phospholipid molecule is any spacer-phospholipid, which can be coupled to a peptide because it contains a functional group capable of reacting with a functional group in the peptide to produce a conjugate according to the present invention. The activated spacer can be an alkylating reagent, such as PEG aldehyde, PEG epoxide or PEG tresylate, or it can be an acylating reagent, such as PEG ester.

Spacers of the present invention comprise, but are not limited to, PEG, poly-amino acids (e.g., poly-glycine, poly-histidine, poly-leucine, polyglutamic acid) and poly-saccharides (e.g., polygalacturonic acid, polylactic acid, polyglycolide, chitin, chitosan), and synthetic polymers, including but not limited to, polyamides, polyurethanes, polyesters. As the person skilled in the art will appreciate, appropriate co-polymers may also be used such as, but not limited to, a co-polymer of poly(methacrylic acid) and N-(2-hydroxy) propyl methacrylamide. Molecules constituting modifications to PEG such as poly (propylene)glycol, are also within the scope of the present invention. Spacer molecules and activation strategies are also disclosed, for example, in U.S. Pat. No. 5,672,662 to Harris et al, which is incorporated herein by reference in its entirety.

The conjugates so obtained may be purified and characterized by appropriate methods.

Irrespective of the method of production, the conjugates may then be presented at the surface of liposomes.

This presentation may be obtained mechanically. Specifically, the conjugates mixed with the liposomes may fit exactly into the phospholipid membrane of the liposomes by means of their lipid doubles chains.

The present invention also relates to the use of this composition in methods of immunization according to the invention.

The present invention therefore relates to a method of immunization comprising a first administration, in particular by injection, of the immunogenic composition according to the invention and a boost administration of said composition (or booster), for example of two successive injections. The boost injections, exposing the same antigen several times, induce a strong secondary immune response. The repeated exposure of peptides derived from the extracellular loops of the P-170 protein, to the immune system, induces immunological memory and-also rapid subsequent secondary responses with a high antibody titer.

More particularly, in the present method of immunization, the injections in humans are carried out 1 month apart.

According to one embodiment, the immunization with the composition according to the invention can be carried out concomitantly with or preceding an anticancer treatment administered to a patient.

Preferably, the immunization precedes the chemotherapy treatment in order to prevent and anticipate the appearance of a multidrug resistance phenotype in the patient. This treatment plan will be preferred to immunization concomitant with the chemotherapeutic treatments when the diagnosis and evolution of the cancer allow the curative therapeutic treatment (chemotherapy) to be delayed by at least 30 days from the date of diagnosis. The late treatment of the cancer does not prevent the method of immunization with the composition of the present invention being used. In fact, combining the anticancer treatment and the immunization with said immunogenic composition at the same time nevertheless induces an immune response for the production of anti-P-170 autoantibodies having a curative or palliative effect on the appearance of the multidrug resistance phenotype. The curative effect of the immunization with the present composition is illustrated by a reversion of multidrug resistance phenotype. In the context of the invention, the expression “reversion” of the multidrug resistance phenotype denotes the change from a phenotype of multidrug resistance to a phenotype “sensitive to the chemotherapeutic treatments”.

For the purpose of the present invention, the term “chemotherapy”, “anticancer” or “chemotherapeutic drugs” is intended to mean any curative or palliative treatment of primary or secondary tumors based on cytotoxic agents. Chemotherapy generally requires several cycles of treatment.

In the context of a method of immunization preceding the chemotherapy treatment, the implementation of the first injection of the composition according to the present invention precedes the start of the chemotherapeutic treatment by at least 60 days, preferably 63 and 67 days.

The compositions according to the invention can be administered topically, systemically (orally, nasally and via other mucosal routes) and/or parenterally (intravenously, subcutaneously, intramuscularly or intraperitoneally) or by combination of these routes, and effectively induce a protective immune response against multidrug resistance. The composition is formulated so as to allow easy administration via the various pathways above. In particular, the choice of the secondary compounds (wetting agent, emulsifier or buffer) is dictated by the chosen mode of administration.

Advantageously, the immunization is carried out by means of intramuscular and intraperitoneal administration, respectively, in humans and in mice.

One of the embodiments of the present invention also relates to the provision of an antibody, in particular an autoantibody, induced against human or murine P-170, which binds specifically to the peptides of the conjugates according to the invention.

This antibody may be a polyclonal or monoclonal antibody, selected from the group comprising the IgG1, IgG2 and IgG3 isotypes and an IgGM. In particular the IgG2 antibody may be of the subtype 2a or 2b.

The present invention also relates to an immunogenic composition comprising, firstly, a carrier and, secondly, conjugates comprising at least one peptide derived from extracellular loop 1 of the P-170 protein allowing the induction of appropriate antibodies, wherein said conjugates exhibiting all or part of the conformation of extracellular loop 1 of the P-170 protein, for the reversion of multidrug resistance appearing in a patient suffering from a cancer.

This composition is particularly suitable for treatments of solid tumors expressing the MDR1 gene encoding the human P-170 protein.

The present invention also relates to the use of the immunogenic composition according to the invention, for producing a vaccine intended for the treatment and/or prevention of a multidrug resistance in a patient suffering from a cancer. The cancer as envisioned will be a kidney cancer, liver cancer, colon cancer, cancer of the intestine, prostate cancer, breast cancer, bladder cancer, brain cancer, blood cancer (leukemia) and/or cancer of the medullary tissues (myeloma). It may also be a solid tumor expressing the MDR1 gene encoding the human P-170 protein. The use of the immunogenic composition may also be carried out in combination with an anticancer treatment.

In particular, the vaccines may be used in patients bearing solid tumors, in particular cancers expressing the MDR1 gene encoding the human P-170 protein, such as kidney cancer, liver cancer, colon cancer, cancer of the intestine, prostate cancer, breast cancer, bladder cancer, brain cancer, blood cancer (leukemia) and/or cancer of the medullary tissues (myeloma). However, as the person skilled in the art will realize, the immunogenic composition according to the present invention may be used for any cancer that is susceptible to chemotherapeutic treatment.

Finally, the composition according to the present invention may be used directly in patients spontaneously expressing multidrug resistance.

The present invention also comprises a method of treatment and/or prevention of a multidrug resistance appearing in a patient suffering from a cancer, such as cancers susceptible to chemotherapeutic treatment, in particular a cancer affecting the kidney, the liver, the colon, the intestine, the prostate, the breast,.the bladder, the brain, the blood (leukemia) and/or the medullary tissues (myeloma), comprising the administration of the immunogenic composition as described.

The cancer particularly targeted by the treatment as described is a solid tumor expressing the MDR1 gene encoding the human P-170 protein.

The invention is also directed at a kit for making use of the immunogenic composition, comprising the immunogenic composition according to the invention and, optionally, reagents and/or instructions for use.

### EXAMPLES

I-1 Preparation of the Conjugates Formed by Covalent Bonding Between the Peptide Region and the Molecules of Fatty Acid Containing a Carbon Chain of Between C12 and C24

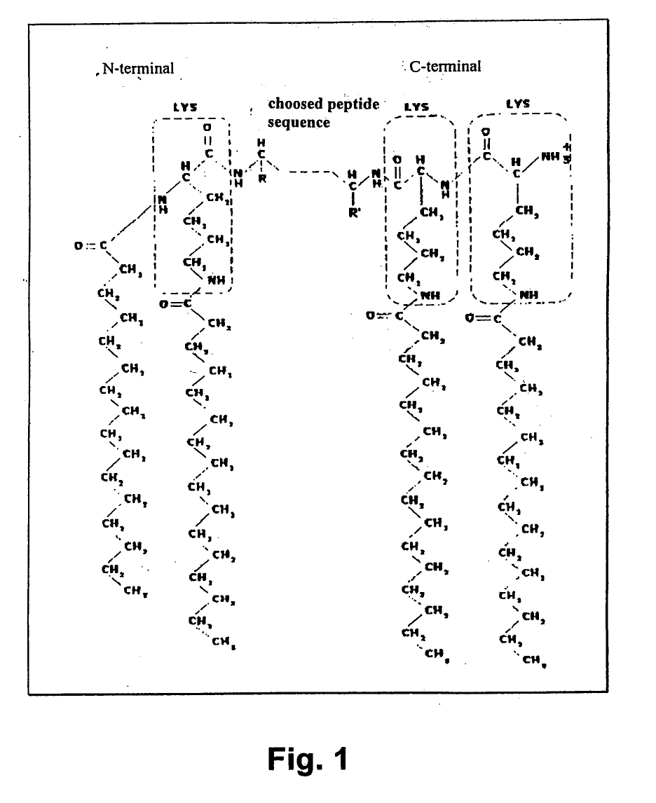

The synthesis of the peptides was carried out at a peptide synthesizer, for example an Applied Biosystem 430A synthesizer, using (13,14)-tert-butyloxycarbonyl/benzyl and, in situ, activation with (N-[(1H-benzotriazol-1-yl)(dimethylamino)methylene]-N-methylmethanaminium hexafluorophosphate N-oxide (Schölzer M. et al. Science 1992, 256 (5054): 221-225) Advanteously, the peptides were then coupled covalently with four molecules of palmitic acid per molecule of peptide (FIG. 1).

An improved method of synthesis allowing more effective production of certain amino acids by avoiding their involvement in early terminating reactions has been developed. This is particularly advantageous in the case of mpp1 due to its length (43 amino acids). In particular, the murine peptides mpp1, mpp2 and mpp4 were synthesized and the results of the synthesis (analysis of the peptide sequence, molecular mass, and purity) were recorded in Table 1 above, each peptide having been controlled for its sequence by amino acid analysis after total acid hydrolysis, for its molecular mass by mass spectrometry analysis and for its purity by HPLC.

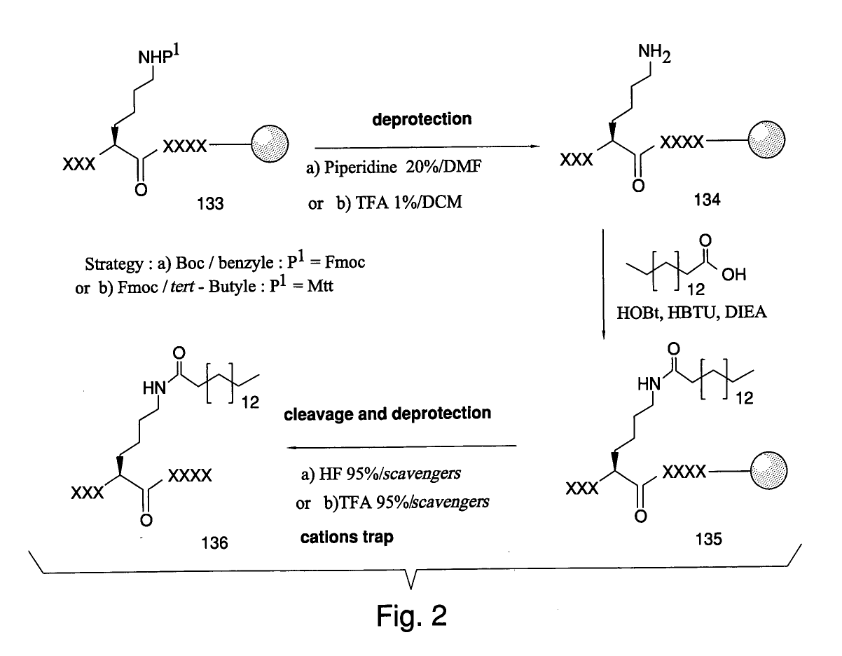

According to this method the synthesis of the conjugates was based on the generation on resin of the desired peptide sequence, and then the deprotection of the amine functions of the N- and C-terminal lysines in order to couple them with the fatty-acid containing a carbon chain of between C12 and C24. The final step was cleavage with anhydrous hydrofluoric acid. The conjugates were obtained using the Boc/benzyl strategy. The optimal approach consisted of generating the peptide sequence on the solid phase and then in coupling an activated fatty acid to a selectively deprotected amine (or thiol) function. If the fatty acid is introduced at the N-terminal end, the peptide does not require any particular functional arrangements. On the other hand, introduction of the fatty acid in the C-terminal position is generally carried out on the E-amino function of a lysine side chain. In the Boc/benzyl strategy, it is often necessary to introduce the Boc-L-Lys(Fmoc)-OH amino acid during synthesis of the peptide. After having generated the entire sequence, the amino function was deprotected and then acylated with the fatty acid containing a carbon chain of between C12 and C24. The conjugate is finally deprotected and cleaved from the resin in the presence of a strong acid, anhydrous hydrofluoric acid (FIG. 2).

I-2 Protocol for the Synthesis of Peptide-PEG-Lipid Conjugates

Solid phase peptide synthesis (SPPS) was advantageously carried out on a 2-chlorotrityl resin preloaded with Gly (H-Gly-2-CITrt, Novabiochem, 0.54 mmol/gr) using the 9-fluorenylmethyloxycarbonyl)/tert butyl (Fmoc/tBU) orthogonal protection strategy. Two Lys residues were incorporated at each N- and C-terminal end of the antigenic peptide sequence produced to allow for the site-specific conjugation of PEG-lipid groups by an amide linkage.

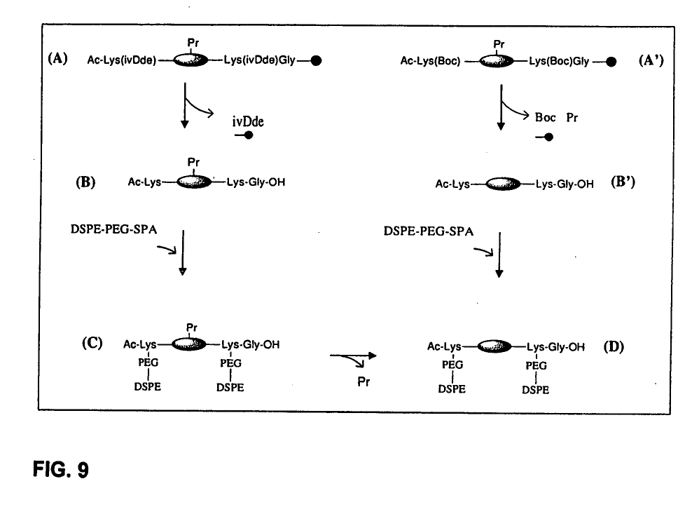

As illustrated in FIGS. 9 (A) to (D), when the antigenic peptide sequence contained internal Lys or His residues, the two terminal Lys residues of the synthesized antigenic peptide sequence were protected with an orthogonal group such as 1-(4,4-Dimethyl-2,6-dioxocyclohexylidene) 3-methyl-butyl (ivDde) (A). Subsequently, the Fmoc group at the N-terminal end of the peptides was removed and the free terminal amino group was acetylated, before chemoselective deprotection of the terminal Lys(ivDde) with 2% hydrazine/dimethylformamide (DMF). Selective resin cleavage of the peptide from the highly acid sensitive 2-chlorotritylresin was achieved with 30% trifluoroethanol (TFE) in dichloromethane, yielding protected peptides bearing free Lys residues at each terminal end (B). Coupling of the partially side chain protected peptides to a pre-activated lipid-PEG ester, such as one formed from the combination of distearoyl phosphatidylethanolamine (DSPE), PEG and the succinimidyl ester of propionic acid (SPA) (DSPE-PEG-SPA) was carried out in solution with dimethylsulfoxide (DMSO) as solvent and in the presence of excess of amine base (C). The reactions were then quenched by the addition of, for example, an excess of ethanolamine. Finally, the side chain protecting groups of the peptide sequence were removed (D) with a standard acidic cocktail, typically trifluoroacetic acid: triisopropyl silane: water (TFA:TIPS:H₂O) (95:2.5:2.5).

As illustrated in FIG. 9A′, if the antigenic peptides did not contain internal Lys or His residues, advantegously tert-butyloxycarbonyl (Boc) groups were used to protect the two terminal Lys residues. Totally deprotected peptides were then obtained by sequential steps of Fmoc N-terminal deprotection, acetylation of the N-terminus and finally simultaneous side-chain deprotection and resin cleavage (B′) with TFA:TIPS:H2O (95:2.5:2.5). Coupling of these non-protected peptides to the activated DSPE-PEG-SPA (D) was carried out under similar conditions as outlined above.

Finally, the peptide-PEG-lipid conjugates obtained this way were then purified by HPLC and their identity confirmed by MALDI-TOF mass spectrometry.

I-3 Evaluation of the Degree of Immunogenicity of the Peptides Coupled to Two or Four Fatty Acids Containing a Carbon Chain of Between C12 and C24

In order to further understand the degree of immunogenicity of the conjugates according to the invention, two types of conjugates were produced and tested. The first type of conjugate corresponded to the synthetic sequences of the peptides derived from the extracellular loops of the murine P-170 protein, covalently coupled to four molecules of fatty acid containing a carbon chain of between C12 and C24, per molecule of peptide. The second type of conjugate was made up of a peptide coupled only to two molecules of fatty acid. This study is illustrated by means of a specific example of dipalmitoylated and tetrapalmitoylated conjugates.

Table III describes the sequences of the di- and tetrapalmitoylated conjugates corresponding to loops 1, 2 and 4 of the murine P-170 protein and also their immunogenic capacity measured through the detection of antibodies by the Dot Blot technique and also the antibody titer by fluorescence units. It was observed that the mice which received liposomes containing conjugates with two palmityl residues did not show an immune response, with the exception of mpp′4, corresponding to loop 4 of the murine P-170 protein. The antibody titer induced by this conjugate was, however, four times lower than the titer induced by mpp4, i.e. the same sequence coupled to four palmityl residues. The mpp4 and mpp2 conjugates engender an immune response with an antibody titer in the region of 400 fluorescence units. In the context of the first peptide synthesis, no antibody was detected following injection of the mpp1 conjugate corresponding to the longest amino acid sequence, which could result from the peptide synthesis. In fact, the synthesis of this loop is very hard and long since there are many possible chemical terminations, or chemical rebridging which could explain the lack of immune response of this first batch of peptides. For the animal immunization trials, a further synthesis of the peptides, in particular of loop 1, was therefore carried out.

In view of these results, the model coupling each molecule of peptides to four molecules of fatty acid containing a carbon chain of between C12 and C24, strengthens the immunogenic capacity of said conjugates incorporated into the membrane of liposomes in the immunogenic composition according to the invention (FIG. 1). The tertiary structure induced by the hydrophobic interactions at the N and C terminal ends therefore plays a role in inducing a substantial and specific humoral immune response. These hydrophobic-type interactions are strong enough to create a loop conformation defined as being the equivalent tertiary structure of the natural structure of the extracellular loops of the P-170 protein. This “natural” conformation is stably exposed at the surface of the carrier, preferably at the surface of the liposomes, in the case of the conjugates coupled to four C12 to C24 fatty acid residues per molecule of peptides. A decrease in the hydrophobic interactions by a factor of two may be insufficient to obtain the “natural” structure resulting in an almost complete inhibition of the antigenic potential of the conjugates according to the invention.

TABLE III

| | | | Detection | |
|---|---|---|---|---|
| | | Number | of | |
| | | of | antibodies | Ac titer |
| | | palmityl | by Dot | fluorescence |
| Conjugate name | Amino acid sequence | chains | Blot | units |
| mpp1 (SEQ ID N0 4) | K-G-GNMTDSFTKAEASILPS | 4 | − | <10 |
| | ITNQSGPNSTLIISNSSLEEE- | | | |
| | G-K-K-NH₂ | | | |
| mpp′1a (SEQ ID NO 21) | G-GNMTDSFTKAEAS-G-K-NH2 | 2 | not tested | not tested |
| mpp′1b (SEQ ID NO 22) | G-LPSITNQSGPNS-G-K-NH2 | 2 | − | <10 |
| mpp′1c (SEQ ID NO 23) | G-TLIISNSSLEEE-G-K-NH2 | 2 | − | <10 |
| mpp′2 (SEQ ID NO 24) | G-KVLTSFTNKELQAYAK-G-K-NH2 | 2 | − | <10 |
| mpp2 (SEQ ID NO 5) | K-G-KVLTSFTNKELQAYAK-G-K-K-NH2 | 4 | + | 360 |
| mpp′4 (SEQ ID NO 25) | G-SRDDDHETKRQNCN-G-K-NH2 | 2 | + | 100 |
| mpp4 (SEQ ID NO 6) | K-G-SRDDDHETKRQNCN-G-K-K-NH2 | 4 | + | 400 |

I-4 Vaccine Preparations

The immunogenic compositions prepared comprise:
Lp1: Mpp conjugates, liposomes (DPMC, DPMG, cholesterol)
Lp2: Liposomes (DPMC, DPMG, cholesterol)
Lp3: Liposomes (DPMC, DPMG, cholesterol), MPLA, conjugates
Lp4: Mpp conjugates.

The liposomes present at their surface the three conjugates mpp1, mpp2 and mpp3 added in a molar ratio of 1:250 with the phospholipids. The organic solvents allowing homogenization of this combination were evaporated off, and the resulting film, after hydration with sterile PBS pH=7.4, was adjusted to a final phospholipids concentration of 4 mM. Finally, the liposomes in suspension are, at the time of immunization, mixed with sterile alum (Pasteur Mërieux) in a ratio by volume. The vaccine preparations injected into the animals therefore correspond to the immunogenic compositions Lp1, Lp2, Lp3 and Lp4 in formulations comprising alum as adjuvant for immunity, the alum also being capable of prolonging the absorption time of the vaccine.

I -5 Animals

The studies were carried out on 6- to 10-week-old female B6D2F1 mice derived from crosses between C57B1/6 females and DBA/2 males (Charles River Laboratories). The mice used in the experiment weigh between 19 and 22 g. Blood samples were taken from the animal 7 to 12 days after immunization, from the retro-orbital sinus.

I-6 Immunization Protocol

The mice were immunized three times, two weeks apart, by intraperitoneal injection with 200 μl of vaccine composition. This experimental protocol is carried out for the four preparations on groups of nine B6D2F1 mice (Iffa Credo, L'Arbresle, France).

To quantify by Dot Blot the various immunoglobulins induced by the immunization, 100 μl of blood were taken, from each mouse, from the retroorbital sinus one day before the booster and 15 days after the final injection. Each blood sample was then centrifuged and the serum isolated was used for the quantification.

I-7 Anticancer Agents

Doxorubicin (Dox) (Sigma) and vinblastine (VLB) were used as anticancer agents in the protocol for the in vivo model of induction of solid tumors and of chemotherapy after immunization.

Doxorubicin is the leading cytostatic antineoplastic agent of the anthracycline family, it is therefore widely used alone or in combination in the treatment of many tumors. The main mode of action of doxorubicin appears to be on the inhibition of DNA topoisomerase II. However, like all anticancer medicinal products, doxorubicin has side effects, in particular of hematological, digestive and inflammatory type, and especially cardiac toxicity, which limits the use thereof in chemotherapeutic treatments. The doxorubicin solution was used at a concentration of 10⁻¹ mol/l the present experimentations.

Vinblastine is a vinca alkaloid commonly used in therapy as an agent for blocking cell mitoses in metaphase, hence its name of mitotic spindle poison. Vinblastine therefore preferentially cures rapidly dividing cells, it is therefore particularly suitable for the treatment of testicular cancer and Kaposi's sarcoma. However, there are many and varying toxic manifestations of vinblastine, the main one being blood toxicity. Finally, the vinblastine solution is used at a concentration of 10⁻² mol/l in the experimental trials of the present invention.

I-8 In vivo Model of Solid Tumor Induction

The B16R cell line, originating from a murine melanoma and selected for its resistance to doxorubicin, was chosen in the present invention for the development of solid tumors. The B16R cells were cultured in vitro, harvested in the exponential growth phase and cleaned with a phosphate buffer saline (PBS) before they were administered by subcutaneous injection into the rear flank of the mouse. The injection volume is 50 μl of a suspension of 1.10⁶ B16R cells in 0.85% NaCl (Candido K A et al. Cancer Res 2001, 61 (1):228-236). The mice developed a melanoma with an average size of 2.0 g±1.2 g within a period of between 22 and 24 days after inoculation of cancerous cells.

After tumor development, the B16R line was confirmed to be resistant to chemotherapeutic treatments with doxorubicin. Consequently, the preceding experimental conditions served as a model for inducing the solid tumor from murine P388R cells (murine lymphoid neoplasma cells characterized and used as reference cells for their MDR properties—Kohls W D. et al. Cancer Res 1986 September, 46(9): 4352-6), also resistant to doxorubicin.

Other tumor cells can be used provided that they exhibit resistance to the chemotherapy tested.

I-9 Protocol for Chemotherapy After Immunization

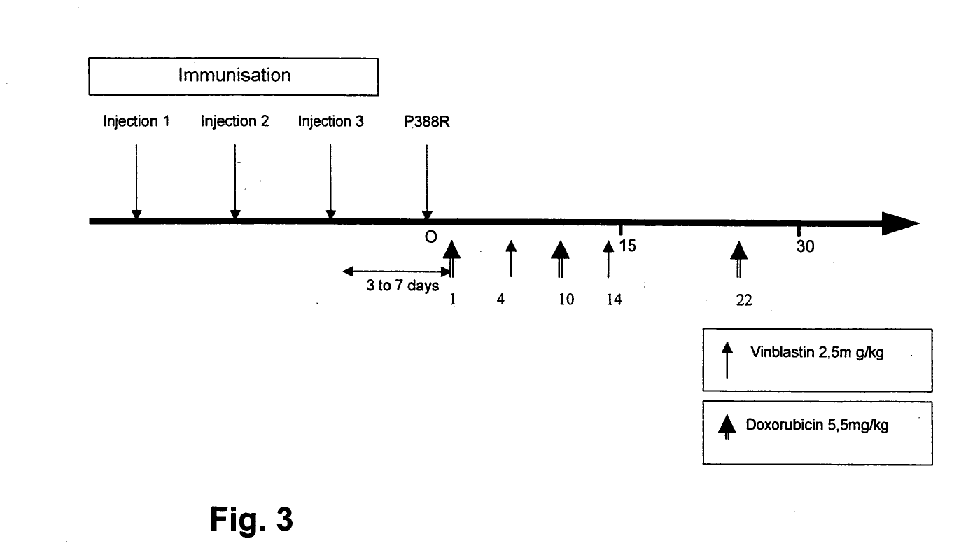

A protocol for chemotherapy using two anticancer agents, vinblastine and doxorubicin, was established as described in FIG. 3. The chemotherapeutic treatment of the mice pre-immunized with the vaccine preparations Lp1 and Lp2 begins one day after the subcutaneous injection of 10⁶ P388R cancer cells (day 0), by means of a weekly injection of doxorubicin at a dose of 5.5 mg/kg (days 1, 10 and 22) followed by the alternating injection of vinblastine at a dose of 2.5 mg/kg (days 4 and 14). During this period, the food intake, water intake and weight of the mice, and also their survival, were recorded. Before the injection with the P388R cells, samples of mouse serum were taken during a period for between 15 and 45 days after immunization, in order to quantify the anti-P170 antibodies and monitor their activity.

I-10 Analysis of the Immune Response by Dot Blot

The conjugates according to the invention serving as antigenic molecules, diluted in PBS, were firstly deposited at ambient temperature onto nitrocellulose membranes. After 30 minutes, these antigenic molecules are blocked with 3 ml of a solution containing PBS-5% skim milk. The membranes were incubated for 2 hours at ambient temperature, without washing, with 24 μl of murine serum prediluted volume-for-volume with PBS, in 2 ml of PBS containing 1% of skim milk and 0.1% of tween 20. Said murine serum was taken during a period of 15 to 45 days after the third immunization. After three washes in PBS-1% skim milk-0.1% tween 20, the membranes were sequentially incubated for 1 hour at ambient temperature, in 3 ml of PBS-1% skim milk-0.1% tween 20 containing the peroxydase anti-mouse secondary antibody diluted to 1/3000, and then after 2 washes, in 3 ml of PBS-1% skim milk-0.1% tween 20. The membranes were then washed once for 10 minutes in PBS alone and kept in a refrigerator at 4° C. overnight, in 500 μl of PBS. A chemoluminescence peroxydase substrate (ECL™ kit, AMERSHAM Pharmacie Biotech) was deposited at the surface of the membranes (0.125 ml/cm²), and left for one minute, and the membranes are then drained off and placed in a “cold” cassette between 2 films of Saran®. The membranes are immediately exposed by autoradiography, the emitted light resulting from the reaction of oxidation of the luminol by the peroxydase being collected on KODAK X-OMAT films with varying times from a few minutes to 1 hour, depending on the strength of the signal. The antibody titers were estimated using a densitometer suitable for the above system. The sensitivity of the chemoluminescence reaction has a threshold for detection of the induced antibodies evaluated at 0.2 ng/ml under the experimental conditions.

The protocols for the experiments to evaluate the immune response in vivo in the mice immunized with the vaccine preparations described above used anti-mpp1, mpp2 and mpp4 antibodies and also Ig (M, G3, G2a, G2b, G1) specific anti-murine secondary antibodies.

I-11 Immune Response in vivo in the B6D2F1 Mice Immunized with the Vaccine Preparations

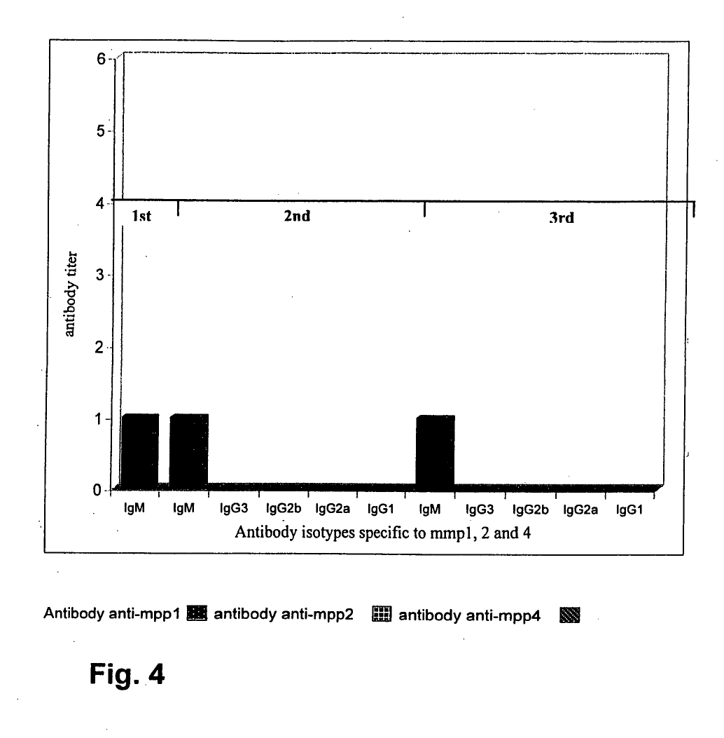

As can be seen from the results obtained and shown in FIG. 4, the immune response of the B6D2F1 mice immunized with the control vaccine Lp2 shows predominantly IgM antibodies. The concentration of the IgM antibodies remains constant in the course of the three immunizations, reflecting an immune response of polyclonal antibodies of a specific type due to the presence of MPLA. The value for the IgM antibodies were subtracted from that found in the sera of mice immunized with the Lp1 vaccine.

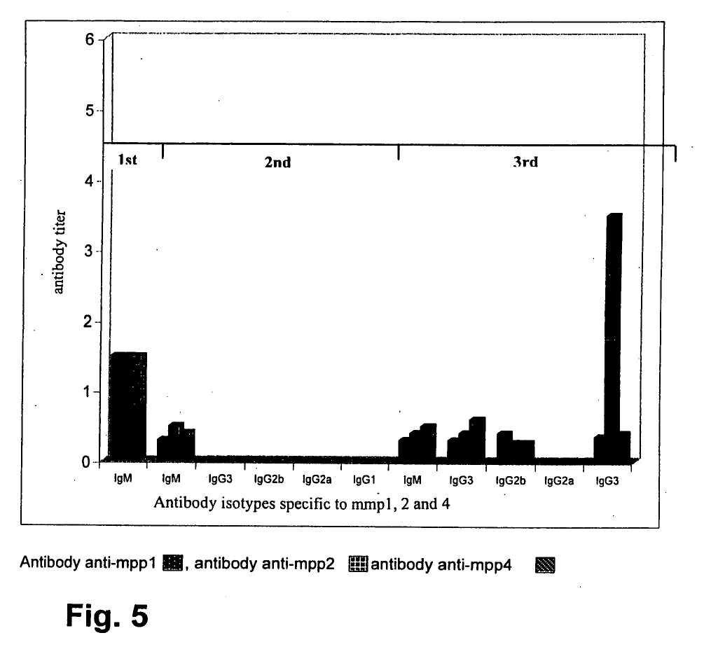

The mice immunized with the vaccine preparation Lp4 exhibit anti-mpp1, anti-mpp2 and anti-mpp3 IgM antibodies following the first immunization. This quantity of IgM antibodies was shown to decrease& until it disappears following the third immunization so as to allow a predominantly IgG1 antibody immune response to emerge (FIG. 5).

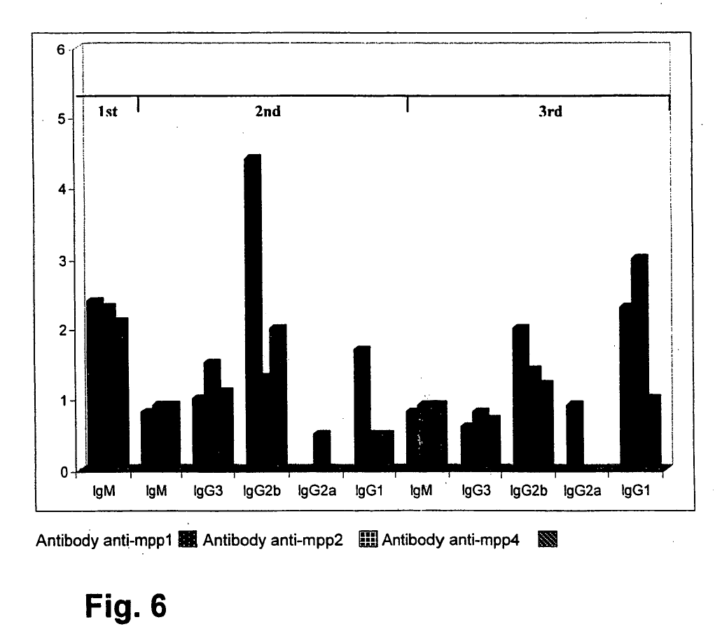

The results shown in FIG. 6 demonstrate that immunization of the mice with the Lp3 vaccine induces the expression of IgM antibodies after the first injection. The IgM antibody titer decreases after the second immunization, during which period the immune response becomes predominantly an IgG2b antibody response. After the third injection, the IgG1 antibody titer is at a maximum; in addition, this immune response is positive for the three conjugates, the mpp2 conjugate being the most immunogenic.

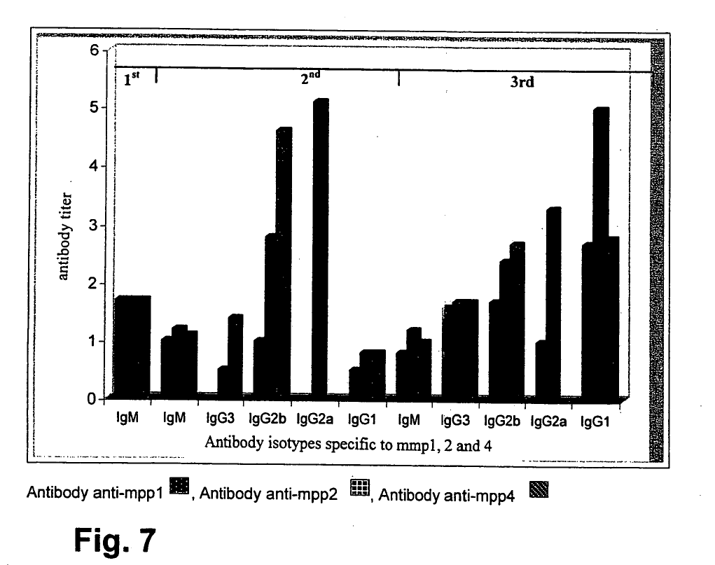

Immunization of the mice with the Lp1 vaccine preparation induces, as supported by the results shown in FIG. 7, that the predominant appearance of an IgM antibody directed against extracellular loops 1, 2 and 4 of the P-170 protein. The quantity of IgM antibody decreases in the course of the second and third immunizations, so as to allow a predominantly IgG anti-mpp2 antibody response to emerge. The IgG3, IgG2a and IgG2b antibody titers are approximately two to three times greater than the basal value, whereas the IgG1 antibody titer is five times greater.

It was observed, by comparing the quantities of IgG1 antibodies, that the mpp2 conjugate is, respectively, 2.6 and 2 times more immunogenic than the mpp1 and mpp4 conjugates. In addition, it is noted that the Lp1 vaccine preparation induces the strongest overall immune response, i.e. the isotypes (IgM, G3, G2a, G2b, G1) corresponding to each of extracellular loops 1, 2 and 4 together.

The antibody titers induced by the mpp1 conjugate are significant and comparable in value to the antibody titers detected for the mpp2 and mpp4 conjugates, unlike the results observed with Tosi et al. (1995 Biochemical and biophysical research communications 212(2): 494-500).

After having determined that the Lp1 vaccine preparation had the best immunogenic capacity, its innocuity was checked over a period of 18 months after the final immunization. The mice immunized exhibit no significant variations in weight compared with the mice immunized with the Lp2 control vaccine. In addition, no behavioral modification, for example with regard to vigilance and appetite, was observed in the animals immunized with the Lp1 vaccine preparation. Finally, histopathological analyses of the organs naturally expressing the P-170 protein (spleen, liver, kidneys, adrenoglands, pancreas, ovaries, heart and lungs) demonstrated a lack of induced toxicity and/or of autoimmunity in the mice immunized with the Lp1 vaccine preparation. Specifically, the only lesions that could be observed in the -intraperitoneal position or at the periphery of the organs (liver, pancreas, spleen and ovaries) were attributed exclusively to the use of alum in the immunogenic composition. Complementary analyses to investigate the agent inducing the lesions observed confirmed this result.

I-12 In vivo Evaluation of the Anti-Chemoresistance Activity Associated with the Immunization with the Lp1 Vaccine Preparation

The in vivo study of the evolution of the multidrug resistance phenotype in the mice immunized with the Lp1 and Lp2 (control) vaccine preparations was initiated according to a protocol of induction of solid tumors followed by the chemotherapeutic treatment plan. The anticancer treatment begins 1 day after inoculation of the cancer cells.

Prior to injection of the P 388R cells into the immunized mice, the antibody titers in the sera of the mice immunized with the Lp1 vaccine preparation were determined: 100%, 40% and 80%, respectively, of the sera exhibited anti-mpp1, 2 and 4 IgG1-type antibodies, with a mean value of 0.3, 0.21 and 0.33 μg/ml (1 U corresponds to 0.2 μg/ml).

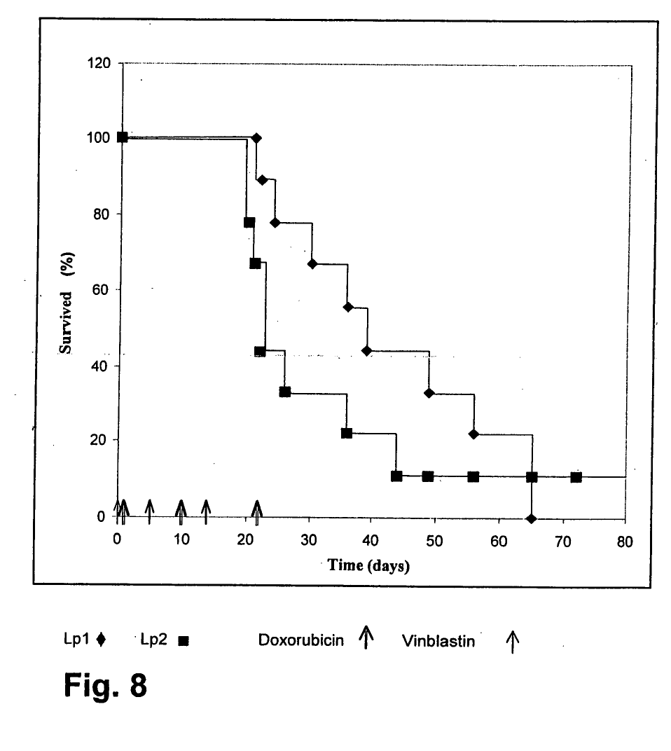

The survival time for the mice immunized with the Lp1 and Lp2 vaccine was represented as a function of time (FIG. 8).

The results represented by graph show that the mean survival time for the group of mice immunized with Lp1 is 39 days, whereas, in the group immunized with the Lp2 preparation, it is 22 days. The mice immunized with the Lp1 vaccine preparation in a preventive manner therefore exhibit a 77% increase in mean survival time compared with the control.

In the Lp2 group, a survival time of 70 days was observed for one of the mice.

This 77% increase in survival time is observed even though the chemotherapeutic treatment as such was only administered 22 days starting from the injection of the resistant cancer cells. Now, it is observed that the survival rate falls starting from the end of administration of the anticancer agents; consequently, it may be deduced therefrom that a complete reversion would be observed if the chemotherapeutic treatment continued, in the knowledge that only the latter has a curative effect, unlike the autoantibodies obtained in the patient immunized with the composition according to the present invention.

These results are very promising since the best published results obtained in the treatment of multidrug resistance with the same cancer model described a 49% increase in survival in mice treated with S9788 at doses of 100 mg/kg/day (Pierré et al. 1992. Invest New Drug. 10: 137-148). In addition, Yang et al. (1999. BBRC. 266: 167-173) observed, with the same cell line, a 35% increase in survival in mice treated with vincristine and cyclosporin A. Other authors have also demonstrated that certain reverting agents such as trans-flupenthixol can accelerate mortality by means of an increase in the invasive potential of the cancer cells.

This increase in survival time is all the more pleasing since the experimental murine cancer models are very demanding with respect to the effectiveness of the treatment, since, as soon as they are inoculated, the cancer cells have a degree of resistance that is greater than that observed clinically. This observation implies that the reversion agents are active as soon as the cancer cells are injected; now, it is noted that these agents are generally active at cytotoxic concentrations that are reached gradually during treatment.

Immunization with the Lp1 vaccine preparation induces in the mice the formation of active autoantibodies capable of rapidly, and in a long-lasting manner, inhibiting, in vivo, resistance to chemotherapy. Immunization with the Lp1 vaccine therefore makes it possible to rapidly inhibit chemoresistance and to reestablish in vivo the activity of anticancer agents in patients who have become refractory to the chemotherapeutic treatment. In the course of complementary experiments, the circulating autoantibodies in the mice immunized with the Lp1 preparation induced no cytotoxicity, no development of autoimmune lesions, nor any increase in the evasive potential of the cancer cells.

The above descriptions use in particular peptides of the murine P170 protein. The protocols described are, however, naturally applicable in a similar manner to the synthesis of any other peptide, in particular those which have been described above for the human P170 protein.

1. A conjugate comprising
at least one peptide based on an extracellular loop of a P-glycoprotein, said peptide having an N- and C-terminal end,
one or more terminal amino acids attached to said N- and C-terminal end of said peptide,
at least two spacer molecules each having two termini,
a first of said termini being covalently attached to a phospholipid, and
a second of said termini being covalently attached to one of said terminal amino acids.

2. The conjugate of claim 1, wherein said at least one peptide has an amino acid sequence that is identical to the amino acid sequence of an extracellular loop of said P-glycoprotein.

3. The conjugate of claim 2, wherein said extracelluar loop is extracellular loop 1, 4 or 6 or sub-loop 1a, 1b or 1c of human P-glycoprotein.

4. The conjugate of claim 2, wherein said amino acid sequence of said peptide is SEQ ID NO. 1, SEQ ID NO. 2, SEQ ID NO. 3, SEQ ID NO. 7, SEQ ID NO. 8, SEQ ID NO. 9, SEQ ID NO. 10, SEQ ID NO. 11, SEQ ID NO. 12 or SEQ ID NO. 13.

5. The conjugate of claim 1, wherein said terminal amino acids are lysine.

6. The conjugate of claim 1, wherein one terminal amino acid is attached to said N- and to said C-terminal end of said peptide.

7. The conjugate of claim 1, wherein said spacer molecules are selected from the group of PEG, poly(propylene) glycol, polylysine, polyglycine, polyglutamic acid, poly-saccharides, synthetic polymers and synthetic co-polymers.

8. The conjugate of claim 1, wherein said phospholipid is phosphatidylethanolamine, and wherein a fatty acid moiety of said phosphatidylethanolamine is a saturated or unsaturated C12-C24 fatty acid.

9. The conjugate of claim 8, wherein said phosphatidylethanolamine (PEA) is phosphatidyidistearoyl PEA, dipalmitoyl PEA, dimyristoyl PEA or di-oleyl PEA.

10. An immunogenic composition comprising
a. at least one conjugate of claim 1 and;
b. at least one liposome or virosome,
wherein administration of an effective amount of said immunogenic composition induces anti-P-glycoprotein antibodies.

11. The immunogenic composition of claim 10, wherein said compounds in (b) is a liposome.

12. The immunogenic composition of claim 10 further comprising an immunomodulator.

13. The immunogenic composition of claim 10, wherein said compound is a liposome and said immunomodulator is lipid A.

14. The immunogenic composition as claimed in claim 11, in which conjugates and the liposomes have a molar ratio of between 1/1 and 1/1000.

15. The immunogenic composition as claimed in claim 14, in which the conjugates and the liposomes have a molar ratio of between 1/1 and 1/250.

16. The immunogenic composition of claim 10, wherein said liposome comprises phospholipid, dimyristoylphosphatidylcholine (DPMC), dimyristoylphosphatidylglycerol (DPMG) and cholesterol.

17. The immunogenic composition of claim 16, wherein DPMC, DPMG and cholesterol are provided at a ratio of 0.9:0.1:0.7.

18. The immunogenic composition of claim 10 comprising at least two or at least three conjugates comprising all or part of the amino acid sequences of at least two extracellular loops or at least three loops of the P-glycoprotein, respectively.

19. The immunogenic composition of claim 18, wherein said at least two conjugates comprise the entire amino acid sequences of at least two extracellular loops of the P-glycoprotein.

20. The immunogenic composition of claim 10 wherein said extracellular loop is extracellular loop 1, 4, 6 of human P-glycoprotein or combinations thereof, or sub-loop 1a, 1b, 1c of human P-glycoprotein or combinations thereof.

21. The immunogenic composition of claim 10 wherein said extracellular loop is extracellular loop 1, 2, 4 of murine P-glycoprotein or combinations thereof.

22. Method for preparing the conjugate of claim 1 comprising:
a. synthesizing an amino acid sequence comprising said at least one peptide based on an extracellular loop of a P-glycoprotein and said one or more terminal amino acid(s) on a solid support, at least one of said terminal amino acid(s) being side chain protected,
b. deprotecting the side chains of said terminal amino acid(s),
c. subsequently, releasing the amino acid sequence from the solid support by cleavage with a-mild acid,
d. coupling a pre-activated spacer-phospholipid molecule- to at least one of said deprotected terminal amino acid(s), and
e. optionally, purifying the conjugate.

23. The method of claim 22, wherein said peptide contains one or more internal amino acids which provide attachment points for spacer-phospholipid molecules.

24. The method of claim 22, wherein deprotection and release conditions in b. and c. do not substantially affect any group protecting a side chain of a non-terminal amino acid.

25. The method of claim 24, wherein the side chains of the terminal amino acid(s) are protected by an orthogonal butyl and the side chains of non-terminal amino acids that provide attachment points for spacer-phospholipid molecules are protected by a benzyl or substituted benzyl group.

26. Method for preparing the conjugate of claim 1 comprising:
a. synthesizing an amino acid sequence comprising said at least one peptide based on an extracellular loop of a P-glycoprotein and said one or more terminal amino acids on a solid support, at least one of said terminal amino acids being side chain protected,
wherein said peptide does not contain internal attachment points for spacer-phospholipid molecules,
b. deprotecting the side chains of terminal amino acid(s),
c. releasing the synthesized amino acid sequence from the solid support by cleavage with a harsh acid,
d. coupling a pre-activated spacer-phospholipid molecule to said deprotected terminal amino acid(s), and
f. optionally, purifying the conjugate.

27. The method of claim 26, wherein (b) and (c) are performed concurrently.

28. A method of treating a patient suffering from cancer for multidrug resistance comprising administering to said patient the immunogenic composition of claim 10 in a multidrug resistance treating or preventing amount.

29. The method of claim 28, wherein an organ affected by said cancer is kidney, liver, colon, intestine, prostate, breast, bladder, brain, blood (leukemia) and/or medullary tissues (myeloma).

30. The method of claim 28, wherein said cancer is a solid tumor expressing a MDR1 gene encoding the human P-glycoprotein.

31. The method of claim 28, wherein said composition is administered in combination with an anticancer treatment.

32. A method of claim 28, wherein said treatment for multidrug resistance constitutes a vaccination against multidrug resistance.

33. A hybridoma which produces:
a monoclonal antibody specifically immunoreactive with the conjugate of claim 1.

34. A monoclonal antibody produced by the hybridoma of claim 33.

35. An immunological assay for detecting P-glycoprotein antigen in a biological sample comprising:
combining the monoclonal antibody of claim 34 with the biological sample; and
assaying the biological sample for antigen binding as a measure of a monoclonal antibody-P-glycoprotein antigen complex formed in said combining step.

36. The immunological assay of claim 35, wherein either the antigen or the monoclonal antibody contains a detectable label.

37. The immunological assay of claim 36 wherein said detectable label is selected from the group consisting of radioactive material, fluorophor, dye, an electron dense compound and an enzyme.

38. A method for the immunological detection of cancer, comprising: combining the monoclonal antibody of claim 34 with a biological sample; and
measuring the amount of monoclonal antibody P-glycoprotein antigen complex formed in the combining step, wherein elevated amounts of said complex indicate the presence of cancer.

39. The method of claim 38 wherein the detections of P-glycoprotein antigen is associated with cancers selected from the group consisting of
kidney, liver, colon, intestine, prostate, breast, bladder, brain, blood (leukemia), medullary tissue (myeloma) cancer and a solid tumor expressing a MDR1 gene encoding the human P-glycoprotein.

40. A diagnostic kit suitable for detecting a P-glycoprotein antigen comprising in containers:
the monoclonal antibody of claim 34 specifically immunoreactive with a P-glycoprotein antigen.
